(19) United States
(12) Patent Application Publication
(10) Pub. No.: US 2024/0115408 A1
(43) Pub. Date: Apr. 11, 2024

(54) Orthosis Device for Animals

(71) Applicant: WIMBA POLAND spolka z ograniczona odpowiedzialnoscia, Krakow (PL)

(72) Inventors: Sylwia KRUZLAK, Bienkowka (PL); Filip ZABAWA, Tarnowiec (PL)

(73) Assignee: WIMBA POLAND spolka z ograniczona odpowiedzialnoscia, Krakow (PL)

(21) Appl. No.: 18/378,620

(22) Filed: Oct. 10, 2023

(30) Foreign Application Priority Data
Oct. 11, 2022 (PL) PL442494

(51) Int. Cl. A61F 5/01

(52) U.S. Cl. CPC A61F 5/0109; A61F 2005/0137; A61F 2005/0144; A61F 2005/0158; A61F 2005/016; A61F 2005/0167; A61F 2005/0172

(57) ABSTRACT

An orthosis device is disclosed, wherein the orthosis includes a frame comprising straps with fasteners, hinges and lateral elements connecting the straps to the hinges, each of which consists of a sleeve with an arcuate shape connected to one lateral element, and a hoop connected to the other lateral element, the hoop being placed in the sleeve with a clearance fit. The orthosis device may be provided with stops provided with grooves, and a rim provided with recesses, with a width corresponding to at least the width of the lateral elements with fit tolerance, and a protrusion situated in the central part of the stop.

### CROSS-REFERENCE TO RELATED APPLICATIONS

This application is a Non-Provisional Patent Application and claims the benefit of and priority to Polish Patent Application No. PL442494, filed on 11 Oct. 2022, and entitled “Orthosis, in particular for animals” the disclosure of which is incorporated by reference as if the same were fully set forth herein.

### TECHNICAL FIELD

The present devices and systems relate generally to orthotic devices which can be used for animals and humans.

### BACKGROUND

Orthotic devices are a significant mean for helping animals and humans suffering various types of injuries or health condition (e.g. knee conditions, broken bones, etc.). Significant work has been performed in the filed of orthotic devices for both humans and animals.

From Chinese patent application no. CN110680590 there is known a knee joint orthosis for pets, and a manufacturing method thereof. By performing three-dimensional scanning and reconstruction of the upper and lower limbs of a dog's knee joint, it is possible to design and manufacture a knee joint orthosis for a dog in a manner adjusted to its needs. Based on the three-dimensional moulding technology, the upper module and the lower module of the joint can better adjust themselves to the four limbs of a dog by cooperation of fastening bandages, due to which it is possible to avoid other negative effects, such as loosening, the dog's discomfort and the like, caused by a difference in shape. The upper module and the lower module of the articulated joint are connected by means of an adjustable set of hinges, due to which the tension of the dog's knee joint can be dispersed, which allows for reducing the load applied to the dog's knee joint. Moreover, the fit between the upper connecting disc and the lower connecting disc can be adjusted so as to realise three modes of motion, namely free rotation, limited rotation and immobilisation; and in this manner, it is possible to satisfy the needs of knee joints of dogs suffering from various diseases.

From American patent application no. US2013253394 A there is in turn known an orthopaedic product for humans having longitudinal spacers, each of which has a tape-coupling element, and a flexible tape mounting the spacers by means of the tape-coupling elements. A semi-rigid and flexibly bent expander is connected to the spacers, and it extends over a specified length therebetween. The expander provides two main positions of the orthopaedic product: stiffening in a straight and bent position of the limb.

On the other hand, from American patent application no. US2014039367 A there is known an orthopaedic device for use on an animal's limb, which provides stability of unstable joints and limbs for short- or long-term support and functioning. This device is designed so as to prevent the occurrence of or reduce the intensity of a joint injury, stabilising the joint, at the same time assisting in or restricting the range of movement. The device can comprise an adjustable tensioning system, which is meant to provide an individual fit for a wide range of animals and injuries.

From application no. WO22046043 A there is also known a knee joint orthosis with adjustable tension, used to relieve a knee joint affected by arthrosis, thus reducing the pain and improving mobility, which consists of: an upper and lower frame connected by a set of relieving hinges, and a set of hinges with adjustable tension, optionally comprising a sensor and enabling remote or automatic tension control of the orthosis.

There is a need for new orthosis/orthotic devices which are inexpensive, easy to manufacture/produce, and which enable both immobilisation of a limb in a joint, as well as its partial or complete bending, according to the medical needs of the patient.

### BRIEF SUMMARY OF THE DISCLOSURE

One of the technical problems tackled by the devices and systems herein is the development of a new orthosis with a simple design which is economical to manufacture, and which enables both immobilisation of a limb in a joint as well as its partial or complete bending, according to the medical needs of the patient.

An object of the invention is an orthosis, in particular for animals, preferably applicable as a metacarpophalangeal joint orthosis, a radiocarpal joint orthosis, an elbow joint orthosis, a glenohumeral joint orthosis, a hip joint orthosis, a knee joint orthosis, a tarsometatarsal joint orthosis, or a tarsometatarsal joint or metatarsophalangeal joint orthosis.

The orthosis according to the invention may include a frame comprising straps with fasteners, hinges and lateral elements connecting the straps to the hinges, each of which consists of a sleeve with an arcuate shape connected to one lateral element, and a hoop connected to the other lateral element. The hoop may be placed in the sleeve and may have a clearance fit. Moreover, the orthosis may include one or more stops provided with grooves, and a rim provided with recesses, with a width corresponding to at least the width of the lateral elements with fit tolerance, as well as a protrusion situated in the central part of the stop.

Preferably, the fastener of the strap consists of an opening on one end of the strap, and a protrusion on the other end thereof.

Preferably, the straps are provided with mounting openings.

Preferably, the straps are provided with pads on the inner side.

Preferably, the stops have the shape of discs.

Preferably, the frame is manufactured entirely as a single flat-printed element.

### BRIEF DESCRIPTION OF THE FIGURES

The accompanying drawings illustrate one or more embodiments and/or aspects of the disclosure and, together with the written description, serve to explain the principles of the disclosure.

FIG. 1 presents an unfolded frame 1 of the orthosis;

FIG. 2 presents a fastened orthosis in a side view, provided with stops 3 and pads 2;

FIG. 3 presents a fastened orthosis provided with stops 3 and pads 2 in an isometric view;

FIG. 4 presents a fastened orthosis provided with stops 3 and pads 2 in a rear view;

FIG. 5 presents a fastened orthosis provided with pads 2 in another embodiment, put on a limb of a dog, without stops 3;

FIG. 6 presents a stop 3 blocking the bending;

FIG. 7 presents a stop 3 blocking the bending, placed in a hinge 6;

FIG. 8 presents a stop 3 restricting the range of bending, placed in a hinge 6;

FIG. 9 presents a stop 3 restricting the range of bending in a side view;

FIG. 10 presents a stop 3 restricting the range of bending in a front view.

### DETAILED DESCRIPTION

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

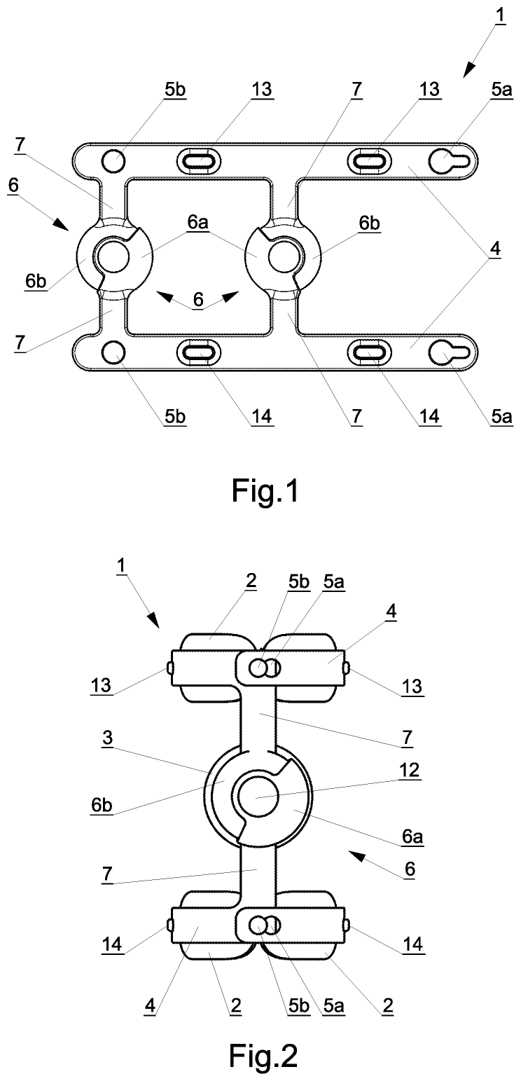

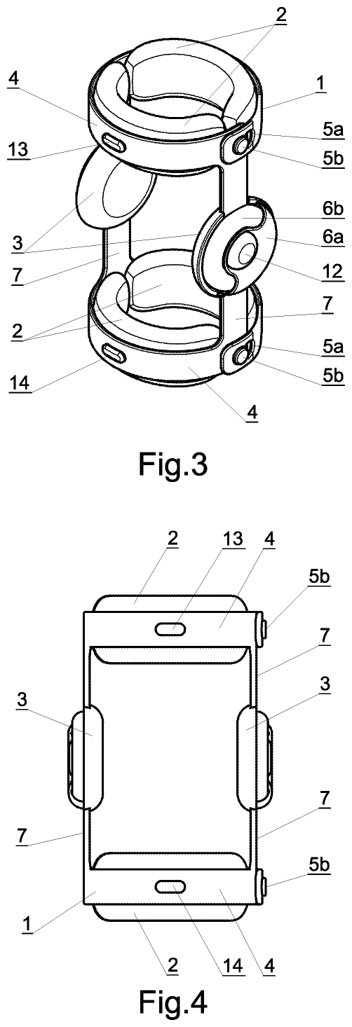

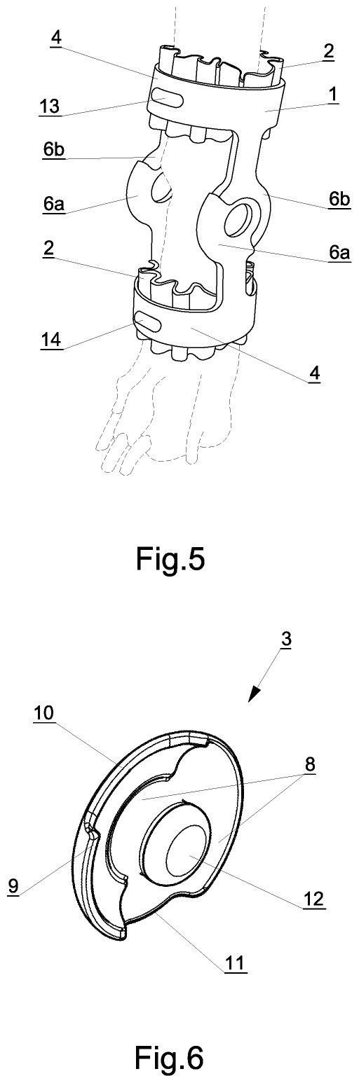

The orthosis according to the invention and as presented in the attached drawings (see e.g. FIGS. 1-5) comprises a frame 1, pads 2 and stops 3. The frame 1 comprises straps 4 with fasteners (consisting of an opening 5a on one end of the strap 4 and a protrusion 5b on the other end thereof), hinges 6 and lateral elements 7 connecting the straps 4 to the hinges 6. FIG. 1 shows an unfolded frame 1 of the orthosis. FIG. 2 shows a fastened orthosis in a side view, provided with stops 3 and pads 2. FIG. 3 shows a fastened orthosis provided with stops 3 and pads 2 in an isometric view. FIG. 4 shows a fastened orthosis provided with stops 3 and pads 2 in a rear view. FIG. 5 shows a fastened orthosis provided with pads 2 in another embodiment, put on a limb of a dog, without stops 3.

The frame 1 may be preferably made using the Multi Jet Fusion (MJF) printing technology from the PA11 material, as a flat element with flexibility enabling the fastening of the straps 4, i.e. bending them so that it would be possible to place the protrusions 5a in the openings 5b (see FIG. 1-2). The straps are further provided with mounting openings 13, 14 used to mount the pads 2 (see FIGS. 2-5). Such form of the frame 1 enables using the orthosis according to its purpose, since in this form the axes of the hinges 6 are arranged parallel to each other and perpendicularly to the axis of the limb placed in the orthosis, and can therefore be bent.

Each of the hinges 6 includes a sleeve 6a with an arcuate shape connected to one lateral element 7, and a hoop 6b connected to the other lateral element 7. The hoop 6b is placed in the sleeve 6a.

The entire frame 1, i.e. the straps 4, the fasteners, the hinges 6 and the lateral elements 7, may be manufactured as a single flat-printed element, and may not/does not require additional connection or assembly of the individual components.

The flexible material from which the frame 1 is made allows for putting the orthosis on and fastening it on a limb. The orthosis is put on an animal's limb so that the hinges 6 are positioned next to the animal's joint, and the straps 4 surround the limb on both sides of this joint (see e.g. FIG. 5).

The inner surface of the straps 4 is provided with a pad 2, preferably soft, printed from the TPU material. The pad can have a shape similar to a cushion, as indicated in FIGS. 2-4, or an accordion, as presented in FIG. 5. The pad 2 allows for putting the orthosis stably on an animal's limb, increases the comfort of usage due to the use of a soft material, and enables a precise fit of the orthosis on the limb.

The stops 3 are described hereinafter with reference to the figures. In FIG. 3, the stops 3 are shown as components of the orthotics, the stops 3 being attached to the hinges 6. FIG. 5 shows an embodiment of an orthotics without the stops 3. FIG. 5 shows holes in the hinges 6 where the stops 3 may be attached.

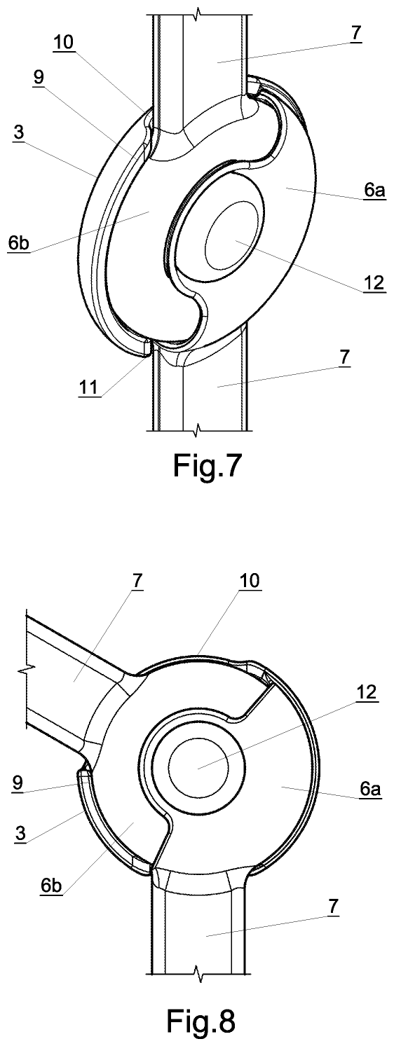

FIG. 6 shows an exemplary embodiment of a stop 3 configured to block the hinges or the bending and/or hinge rotation at the hinges. When attached to the hinges 6, a stop 3 may block the hinges 6 and the bending of the foot/limb on which the orthotics is attached. FIG. 7 shows an exemplary embodiment of a stop 3 attached to/placed into a hinge 6. The stop 3 may block the bending at the hinges 6.

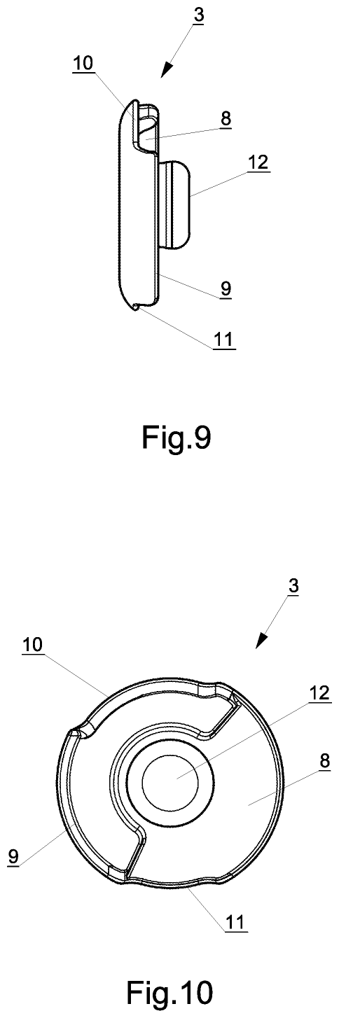

FIG. 8 shows an exemplary embodiment of a stop 3 attached to/placed into a hinge 6. The stop 3 is configured to restrict the range of bending and/or hinge rotation at the hinge 6. FIG. 9 shows a side view of an exemplary embodiment of a stop 3 configured to restrict the range of bending and/or range of hinge rotation. FIG. 10 shows a front view of an exemplary embodiment of a stop 3 configured to restrict the range of bending and/or range of hinge rotation.

The stops 3 may or preferably have the form of discs provided with grooves 8 on at least one of their surfaces, and their edges are provided with a rim 9 provided with recesses 10, 11 with a width corresponding to at least the width of the lateral elements 7 with fit tolerance. Depending on the degree of exposing the edge of the hinge 6, the lengths of the recesses 10, 11 in the rim 9 comprising or attached on the hinge 6 affect the range of its movement or the angular range of the hinge, meaning the longer the recesses 10, 11, the wider the range of movement of the hinge 6.

In an exemplary embodiment, the rim 9 of stop 3 may include a recess 10 extending over a relatively large circular section (e.g. 90 degree angle) of the disc (when stop 3 has a disc shape) thereby allowing the movement/rotation of the hoop 6b (and the lateral element 7 connected with the hoop 6b) with respect to the sleeve 6a (and the lateral element 7 connected with the sleeve 6a) over a relatively large angular range. The rotation angle may refer herein to the angle of rotation of the hoop 6a with respect to the sleeve 6b (and implicitly the angle between the lateral element 7 attached to hoop 6a and lateral element 7 attached to sleeve 6b).

In an exemplary embodiment, the rim 9 of stop 3 may include a recess 10 extending over a relatively small circular section (e.g. 30 degree angle) of its disc thereby allowing the movement/rotation of the hoop 6b (and the lateral element 7 connected with the hoop 6b) with respect to the sleeve 6a (and the lateral element 7 connected with the sleeve 6a) over a relatively small angular range.

In an exemplary embodiment, the rim 9 of stop 3 may include a recess 10 extending over essentially the smallest circular disc section that accommodates the lateral element 7 connected with the hoop 6b. In such a case, the rim 9 blocks the movement of the hoop 6b (and the lateral element 7 connected with the hoop 6b) with respect to the sleeve 6a and the element 7 attached to the sleeve 6a. In such a case, the element 7 connected to the hoop 6 fits into the recess 10 without any room for movement/rotation (i.e. the width of lateral element 7 is essentially equal with the width of the recess).

In an exemplary embodiment, the rim 9 of stop 3 may include a recess 11 extending over a circular section so as to accommodate the lateral element 7 attached to the sleeve 6a and to block the movement of the stop 3 with respect to the lateral element 7 attached to the sleeve 6a (i.e. the width of lateral element 7 is essentially equal to the width of the recess).

In an exemplary embodiment the stop 3 may include a recess 11 extending over a circular section so as to accommodate the lateral element 7 attached to the sleeve 6a and to allow some movement (over a certain angular range) of the stop 3 with respect to the lateral element 7 attached to the sleeve 6a.

In the central part of the stop 3 there is or may be a protrusion 12 allowing for immobilisation/attachment of the stop 3 in the hinge 6. When stop 3 is put on or attached to the hinge 6, the protrusion 12 passes through the centre of the hinge axis, preferably deeper than half of its cross-section. The grooves 8 allow for placing the stop 3 in the hinge 6; the recesses 10, 11 in the rim 9 prevent bending, or restrict its range. By various degrees of fits (from clearance to interference), the stops 3 can also increase the resistance accompanying the bending of the hinge 6, which can be desired when healing certain dysfunctions.

The stops 3 are preferably made using the MJF printing technology from PA12 (colour printing), PA11 (high stiffness and durability) and TPU materials (using the friction and flexibility of the material). Due to the various developed designs of the stops 3, in particular due to the ability to use various lengths and placements of the recesses 10, 11, it is possible to use the orthosis for various purposes, both for completely stiffening the joint, with simultaneous control of its bending angle, as well as for dysfunction therapy, by controlling the range of an animal's movement, as well as controlling the resistances which the orthosis presents to the animal, which is achieved through various degrees of fits of the stops 3 (from clearance to interference).

By adjusting the length of the rim 9 and recesses 10, 11 it is possible to design stops 3 blocking the bending or restricting the bending/rotation angles to certain rotation angles. The rotation angle may refer herein to the angle of rotation of the hoop 6a with respect to the sleeve 6b (and implicitly the angle between the lateral element 7 attached to hoop 6a and lateral element 7 attached to sleeve 6b).

In an exemplary embodiment, it is disclosed a first-stop 3 which includes a recess 10 extending over a circular-section of its disc, the circular-section having a first-length, thereby allowing the movement/rotation of the hoop 6b (and the lateral element 7 connected with the hoop 6b) with respect to the sleeve 6a (and the lateral element 7 connected with the sleeve 6a) over a first-angular-range.

In an exemplary embodiment, it is disclosed a second-stop 3 which includes a recess 10 extending over a circular-section of its disc, the circular-section having a second-length, thereby allowing the movement/rotation of the hoop 6b (and the lateral element 7 connected with the hoop 6b) with respect to the sleeve 6a (and the lateral element 7 connected with the sleeve 6a) over a second-angular-range. The second-length is different from the first-length and the second-angular-range is different from the first-angular-range. The second-angular-range may be equal to zero, meaning that the second-stop may block the movement/rotation of the hinge 6.

In an exemplary embodiment it is disclosed an orthotic system including a frame 1, one or more pads 2 and one or more stops 3. The one or more stops may include a first-stop and a second-stop. The orthotic system enables an user to use either the first-stop in a first situation (e.g. when some mobility/large hinge angular rotation is desired) or the second-stop (e.g. when the mobility needs to be restricted significantly, such as when the hinge is blocked or a small rotation angle is permitted). The orthotic system may be sold as a kit which may include an array of stops and pads thereby allowing the user to customize the orthotic device to the specific needs of the animal.

Injuries of the lower limbs of small animals often require therapy using an orthosis or stiffening. The orthosis according to the invention can be used on the lower part of a front or hind leg, in order to provide its stability and support. The orthoses can be available in a full range of sizes adjusted to a specific animal, in order to provide comfort and effectiveness of use. The orthoses according to the invention can be found useful in helping to cure wrist hyperextension, radiocarpal joint instability, arthrosis, a damaged lateral wrist ligament, as well as for postoperative support of wrist arthrodesis or its failure, and can also be used in prophylaxis as support during amputation of a contralateral limb, during foot injuries, including a damaged tendon and amputation of the toes, during peripheral neuropathy and distal neuropathy of the brachial plexus. The frame 1 of the orthosis has a wide range of uses, depending on the type of the used stops 3. The orthosis can be made in particular in the 3D printing technology, in which the frame 1 is made of a single component in the same technological process. Similarly, the pads 2 and the stops 3 are made of a single material. The orthosis according to the invention is preferably manufactured on the basis of a limb scan, with no need to make physical casts or shells, e.g. made from plaster. The frame 1 of the orthosis according to the invention is printed as a flat element, which when bent in order to be put on a limb allows for movement in the hinges 6. The orthosis according to the invention can be made in a plurality of sizes, including individually, according to the dimensions of a specific animal. Adjustment of the orthosis to the dimensions of a specific animal is achieved by adjusting the length of the straps 4 or the lateral elements 7. According to international tolerance classes which are established in ISO 286, the scope of variance of the dimensions of individual elements of the orthosis corresponds to class IT 13.

While various aspects have been described in the context of a preferred embodiment, additional aspects, features, and methodologies of the claimed systems will be readily discernible from the description herein, by those of ordinary skill in the art. Many embodiments and adaptations of the disclosure and claimed systems other than those herein described, as well as many variations, modifications, and equivalent arrangements and methodologies, will be apparent from or reasonably suggested by the disclosure and the foregoing description thereof, without departing from the substance or scope of the claims.

What is claimed is:

1. An orthotic device comprising:
a frame further comprising:
an upper-strap configured to be strapped on or around an upper-part of a limb of a subject;
an upper-lateral-element connected to the upper-strap;
a hoop-hinge-part connected to the upper-lateral-element;
a lower-strap configured to be strapped on or around a lower-part of the limb of the subject;
a lower-lateral-element connected to the lower-strap;
a sleeve-hinge-part connected to the lower-lateral-element, wherein the hoop-hinge-part is disposed inside the sleeve-hinge-part thereby forming a hinge; and
a stop-part disposed inside a hole formed at the center of the hinge, the stop-part being configured to restrict the rotation of the hoop-hinge-part in the sleeve-hinge-part;
wherein the hoop-hinge-part is configured to rotate in the sleeve-hinge-part, in the absence of the stop-part or if the rotation is not restricted by the stop-part, thereby allowing the upper-lateral-element to rotate around the hinge with respect to the lower-lateral-element.

2. The orthotic device of claim 1 wherein the stop-part has a disc shape comprising a rim, wherein the rim further comprises a first-recess configured to accommodate the upper-lateral-element, wherein the first-recess has a first-recess-length and wherein the upper-lateral-element can rotate with respect to the lower-lateral-element and with respect to the stop-part along the first-recess-length between the two ends of the first-recess.

3. The orthotic device of claim 2, wherein the rim further comprises a second-recess configured to accommodate the lower-lateral-element, wherein the second-recess has a second-recess-length.

4. The orthotic device of claim 3, wherein the second-recess-length is equal to a width of the lower-lateral-element and wherein the lower-lateral-element cannot rotate with respect to the stop-part.

5. The orthotic device of claim 2, wherein the first-recess-length is equal to a width of the upper-lateral-element and wherein the upper-lateral-element cannot rotate with respect to the stop-part and the lower-lateral-element thereby immobilizing the limb.

6. The orthotic device of claim 1, wherein the stop-part further comprises a protrusion configured to pass through the center of the hinge and to attach the stop-part to the hinge.

7. The orthotic device of claim 1, wherein the upper-strap further comprise fasteners configured to attach one end of the upper-strap to another end of the upper-strap.

8. The orthotic device of claim 1, further comprising one or more pads attached to the upper-strap and the lower-strap wherein the pads provide for a comfortable attachment interface between the straps and the limb of the subject.

9. The orthotic device of claim 1, wherein the frame is manufactured as a single flexible flat element which can be bent so as to allow the upper-strap and the lower-strap to be wrapped around the limb.

10. The orthotic device of claim 9, wherein the frame is printed by Multi Jet Fusion printing technology.

11. An orthotic system comprising:
a frame further comprising:
an upper-strap configured to be strapped on or around an upper-part of a limb of a subject;
an upper-lateral-element connected to the upper-strap;
a hoop-hinge-part connected to the upper-lateral-element;
a lower-strap configured to be strapped on or around a lower-part of the limb of the subject;
a lower-lateral-element connected to the lower-strap;
a sleeve-hinge-part connected to the lower-lateral-element;
wherein the hoop-hinge-part is disposed inside the sleeve-hinge-part thereby forming a hinge;
a first-stop-part configured to be disposed inside a hole formed at the center of the hinge, the first-stop-part being configured to restrict the rotation of the hoop-hinge-part in the sleeve-hinge-part; and
a second-stop-part configured to be disposed inside a hole formed at the center of the hinge, the second-stop-part being configured to restrict the rotation of the hoop-hinge-part in the sleeve-hinge-part;
wherein the second-stop-part provides a different restriction to the rotation of the hoop-hinge-part in the sleeve-hinge-part than the restriction provided by the first-stop-part.

12. The orthotic system of claim 11, wherein the first-stop-part allows a rotation of the hoop-hinge-part in the sleeve-hinge-part by a first-rotation-angle and wherein the second-stop-part allows a rotation of the hoop-hinge-part in the sleeve-hinge-part by a second-rotation-angle which is different from the first-rotation-angle.

13. The orthotic system of claim 12, wherein the first-rotation-angle corresponding to the first-stop-part is essentially equal to zero thereby blocking rotation and immobilizing the limb, and wherein the second-rotation-angle corresponding to the second-stop-part is different from zero.

14. The orthotic system of claim 11, wherein the first-stop-part is used for a certain medical needs of the limb of the subject whereas the second-stop-part is used for another medical need of the limb of the subject.

15. Orthosis comprising a frame comprising straps with fasteners and hinges, characterized in that the frame (1) comprises straps (4) with fasteners, hinges (6) and lateral elements (7) connecting the straps (4) to the hinges (6), each of which consists of a sleeve (6a) with an arcuate shape connected to one lateral element (7), and a hoop (6b) connected to the other lateral element (7), the hoop (6b) being placed in the sleeve (6a) with a clearance fit; moreover, the orthosis is provided with stops (3) provided with grooves (8), and a rim (9) provided with recesses (10, 11), with a width corresponding to at least the width of the lateral elements (7) with fit tolerance, and a protrusion (12) situated in the central part of the stop (3).

16. The orthosis according to claim 15, characterized in that the fastener of the strap (4) consists of an opening (5a) on one end of the strap (4), and a protrusion (5b) on the other end thereof.

17. The orthosis according to claim 15, characterized in that the straps (4) are provided with mounting openings (13, 14).

18. The orthosis according to claim 15, characterized in that the straps (4) are provided with pads (2) on the inner side.

19. The orthosis according to claim 15, characterized in that the stops (3) have the shape of discs.

20. The orthosis according to claim 15, characterized in that the frame (1) is manufactured entirely as a single flat-printed element.
